(19) United States
(12) Patent Application Publication
(10) Pub. No.: US 2025/0356489 A1
(43) Pub. Date: Nov. 20, 2025

(54) MEDICAL IMAGE PROCESSING SYSTEM, METHOD, AND APPARATUS

(71) Applicant: TERUMO KABUSHIKI KAISHA, Tokyo (JP)

(72) Inventor: Yuji ONIMURA, Fujinomiya Shizuoka (JP)

(21) Appl. No.: 19/286,137

(22) Filed: Jul. 30, 2025

Related U.S. Application Data
(63) Continuation of application No. PCT/JP2024/002056, filed on Jan. 24, 2024.

(30) Foreign Application Priority Data
Jan. 30, 2023 (JP) 2023-011976

(51) Int. Cl. G06T 7/00; G16H 30/20; G16H 30/40

(52) U.S. Cl. CPC G06T 7/0012; G16H 30/20; G16H 30/40; G06T 2207/10132; G06T 2207/30021; G06T 2207/30101

(57) ABSTRACT

A medical image processing system includes a rotatable catheter insertable into a blood vessel and including at least one of: an ultrasound transceiver configured to transmit ultrasound waves and receive the waves reflected by the blood vessel and an optical transceiver configured to emit near infrared rays and receive the rays reflected by the blood vessel, and a processor configured to generate, based on the ultrasound waves or the near infrared rays, a series of images each showing a location of a boundary of the blood vessel at a rotation angle of the transceiver, generate meta-information about the series of images, embed the meta-information in the series of images, and generate a cross-sectional image of the blood vessel showing the boundary using the series of images with the meta-information, and output the generated cross-sectional image.

### CROSS-REFERENCE TO RELATED APPLICATION

This application is a continuation of International Patent Application No. PCT/JP2024/002056 filed Jan. 24, 2024, which is based upon and claims the benefit of priority from Japanese Patent Application No. 2023-011976, filed Jan. 30, 2023, the entire contents of which are incorporated herein by reference.

### TECHNICAL FIELD

Embodiments described herein relate to a medical image processing system, method, and apparatus.

### BACKGROUND ART

When performing a catheter treatment like percutaneous coronary intervention (PCI), a patient's blood vessel is imaged using multiple methods at the same time, such as intravascular ultrasound (IVUS), optical coherence tomography (OCT), and angiography (Angio). However, these different imaging methods are typically not synchronized. This means that when viewing the images, medical staff need to mentally align them with respect to landmarks such as a branching vessel or an implanted stent. This process is inconvenient and has room for improvement.

For example, there is a known inspection data processing apparatus that synchronously displays a plurality of pieces of inspection data of a patient.

### SUMMARY

Embodiments of this disclosure provide a medical image processing system, method, and apparatus capable of embedding, in an image, information necessary for image synchronization.

According to one aspect, a medical image processing system comprises a catheter rotatable and insertable into a blood vessel and including at least one of: an ultrasound transceiver configured to transmit ultrasound waves and receive the waves reflected by the blood vessel in a radial direction of the catheter when the catheter is inserted in the blood vessel, and an optical transceiver configured to emit near infrared rays and receive the rays reflected by the blood vessel in the radial direction when the catheter is inserted in the blood vessel; a memory; and a processor configured to execute a program that is stored in the memory to: generate, based on the ultrasound waves received by the ultrasound transceiver or the near infrared rays received by the optical transceiver, a series of images each showing a location of a boundary of the blood vessel at a particular rotation angle of the ultrasound transceiver or the optical transceiver, generate meta-information about the series of images, embed the generated meta-information in the series of images, and generate a cross-sectional image of the blood vessel showing the boundary using the series of images with the meta-information, and output the generated cross-sectional image.

According to one aspect, information necessary for image synchronization with another apparatus can be embedded in an image.

### BRIEF DESCRIPTION OF DRAWINGS

FIG. 1 is an explanatory diagram illustrating a configuration of an image diagnostic system.

FIG. 2 is a block diagram illustrating a configuration of an image processing apparatus.

FIG. 3 is an explanatory diagram illustrating an outline of an embodiment.

FIG. 4 is an explanatory diagram regarding meta-information adding processing.

FIG. 5 is a flowchart of a processing procedure executed by the image processing apparatus.

### DESCRIPTION OF EMBODIMENTS

Embodiments of this disclosure will be described below in detail with reference to the drawings.

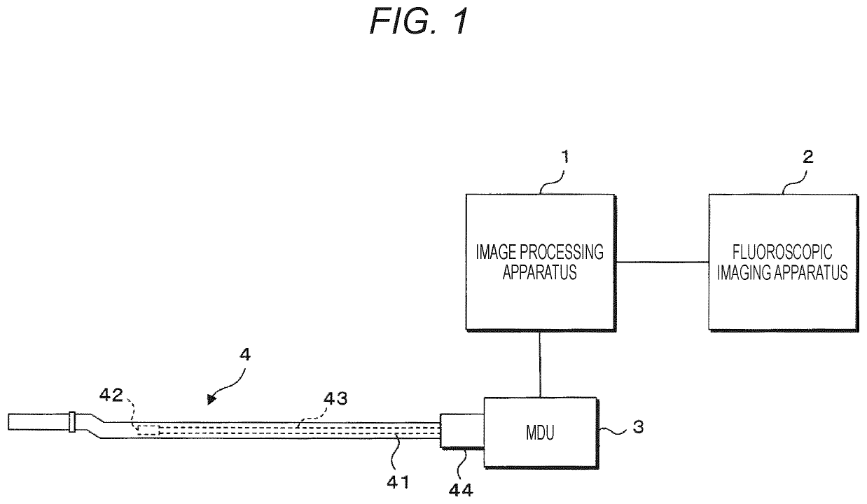

FIG. 1 is an explanatory diagram illustrating a configuration of an image diagnostic system according to an embodiment. The present embodiment will describe an image diagnostic system that synchronously displays blood vessel images captured by a plurality of devices or modalities. The image diagnostic system includes an image processing apparatus 1 and a fluoroscopic imaging apparatus 2. The apparatuses are communicably connected according to a standard such as a local area network (LAN) or a high-definition multimedia interface (HDMI) (registered trademark).

Note that the blood vessel is an example of a luminal organ, and the luminal organ to be imaged is not limited to the blood vessel, and may be another luminal organ such as a bile duct, a pancreatic duct, a bronchus, or an intestine.

The image processing apparatus 1 generates a cross-sectional image of a blood vessel of a subject or a patient using a dual type image diagnosis catheter 4 having both IVUS and OCT functions. The catheter 4 is connected to the image processing apparatus 1 via a motor drive unit (MDU) 3. The image processing apparatus 1 is connected to the MDU 3 through an interface circuit.

The present embodiment has described the case where the catheter 4 is of a dual type having both IVUS and OCT functions, but the catheter 4 may have only one of the IVUS function and the OCT function.

The catheter 4 is a medical instrument to be inserted into the blood vessel of the subject, and includes a sheath 41, a sensor unit 42, a drive shaft 43, and a connector unit 44. The connector unit 44 is a connector that connects the catheter 4 to the MDU 3. In the following description, a side far from the connector unit 44 of the catheter 4 will be referred to as a distal side, and a side of the connector unit 44 will be referred to as a proximal side.

The sheath 41 is a tube to be inserted into the blood vessel. The sensor unit 42 includes one or more sensors that transmit and receive ultrasound waves and near infrared rays for generating cross-sectional images. The sensor unit 42 includes an ultrasound transmitter and receiver (or an ultrasound transceiver) that transmits and receives ultrasound waves, and an optical transmitter and receiver (or an optical transceiver) that emits and receives near infrared rays. The drive shaft 43 is a shaft inserted into the sheath 41 and has a distal end to which the sensor unit 42 is connected. The sensor unit 42 and the drive shaft 43 are configured to be rotatable and movable forward and backward inside the sheath 41.

The ultrasound transmitter and receiver and the optical transmitter and receiver respectively emit ultrasound waves and near infrared rays in the radial direction based on an encoder pulse signal (hereinafter also referred to as encoder information) output from the MDU 3. The image processing apparatus 1 acquires line data indicating the intensity with respect to a distance from the ultrasound transmitter and receiver and the optical transmitter and receiver on the basis of the reflected waves of the ultrasound waves and near infrared rays. The image processing apparatus 1 generates a cross-sectional image (i.e., an ultrasound cross-sectional image and an optical coherence cross-sectional image) of the blood vessel based on the acquired line data.

The MDU 3 is a drive device to which the catheter 4 is detachably attached, and controls the longitudinal and rotational motions of the imaging core (i.e., the sensor unit 42 and the drive shaft 43) of the catheter 4 inserted into the blood vessel by driving a built-in motor according to operation performed by the user.

The fluoroscopic imaging apparatus 2 is a device for capturing a fluoroscopic image of the inside of the patient body, and includes, for example, an angiography apparatus that performs an angiographic examination. The fluoroscopic imaging apparatus 2 includes an X-ray source and an X-ray sensor and generates a fluoroscopic X-ray image of a patient by the X-ray sensor receiving X-rays emitted from the X-ray source. A radiopaque marker (not illustrated) is attached to the distal end of the catheter 4, so that the position of the catheter 4 is visible in the fluoroscopic image.

In the present embodiment, the image processing apparatus 1 and the fluoroscopic imaging apparatus 2 synchronously display images. Specifically, the image processing apparatus 1 embeds meta-information indicating an image acquisition timing in a cross-sectional image of a blood vessel generated by the image processing apparatus 1 itself, transfers the cross-sectional image to the fluoroscopic imaging apparatus 2, and causes the fluoroscopic imaging apparatus 2 to synchronously display the cross-sectional image. The process thereof will be described later in detail.

Note that, in the image diagnostic system according to the present embodiment, the image processing apparatus 1 and the fluoroscopic imaging apparatus 2 are assumed to be directly connected, but the present embodiment is not limited thereto. For example, a personal computer or the like may be connected between the image processing apparatus 1 and the fluoroscopic imaging apparatus 2, and the computer may relay the image processing apparatus 1 and the fluoroscopic imaging apparatus 2.

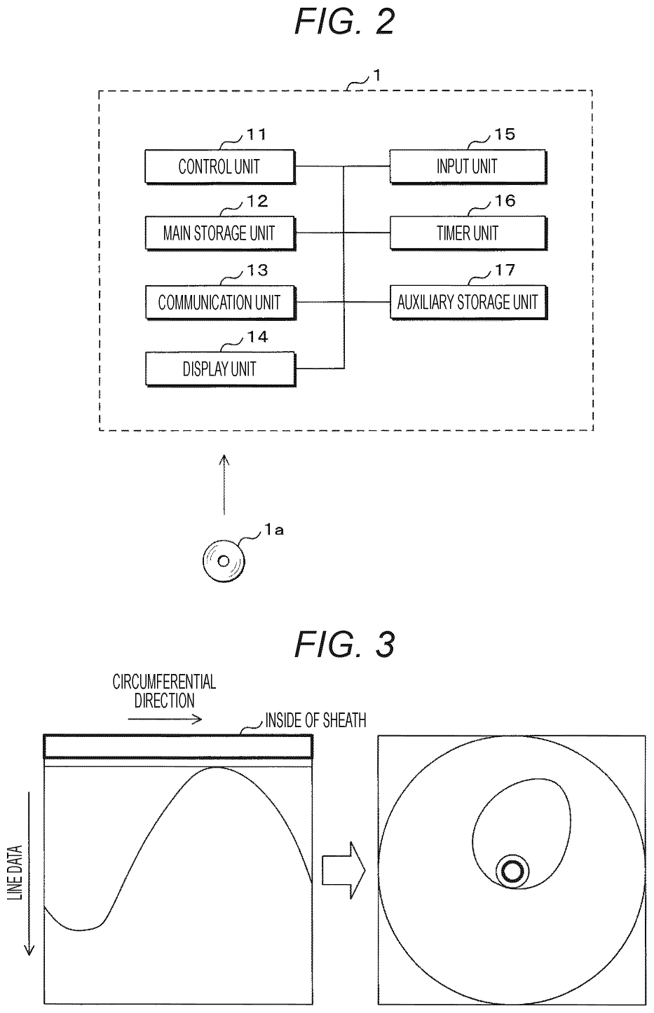

FIG. 2 is a block diagram illustrating a configuration of the image processing apparatus 1. The image processing apparatus 1 includes a control unit 11, a main storage unit 12, a communication unit 13, a display unit 14, an input unit 15, a timer unit 16, and an auxiliary storage unit 17.

The control unit 11 includes one or more arithmetic processing units, such as a central processing unit (CPU), a micro-processing unit (MPU), and a graphics processing unit (GPU). In addition, the control unit 11 performs various information processes, control processes, and some other processes by loading and executing programs stored in the auxiliary storage unit 17. The main storage unit 12 is a temporary memory area such as a static random access memory (SRAM) or a dynamic random access memory (DRAM) and temporarily stores data necessary for the control unit 11 to execute arithmetic processing. The communication unit 13 is a communication module that performs processing related to communication and transmits and receives information to and from the outside. The display unit 14 is a display screen such as a liquid crystal display, and displays an image. The input unit 15 is an operation user interface such as a keyboard and a mouse, and receives an operation input from a user. The timer unit 16 is a timer that measures the current time. The auxiliary storage unit 17 which is a non-volatile storage area, such as a large-capacity memory or a hard disk, stores programs needed by the control unit 11 to perform processes and other data.

Note that the image processing apparatus 1 may further include a reading unit that performs reading operations on a portable storage medium 1a such as a compact disk (CD)-ROM or a digital versatile disc (DVD)-ROM, and thus, may read a program from the portable storage medium 1a and then may execute the program.

FIG. 3 is an explanatory diagram illustrating an outline of the embodiment. With reference to FIG. 3, the outline of the present embodiment will be described below.

FIG. 3 illustrates, on the left side, an ultrasound cross-sectional image of a polar coordinate system in which line data indicating the intensity with respect to the distance from the ultrasound transmitter and receiver is arranged in the lateral direction. For example, the ultrasound transmitter and receiver of the catheter 4 transmits ultrasound waves 512 times during one rotation of the sensor unit 42. In this case, the ultrasound transmitter and receiver receives the reflected wave 512 times in one rotation. The ultrasound transmitter and receiver transmits the received 512 signals to the image processing apparatus 1 via the MDU 3. The image processing apparatus 1 performs predetermined arithmetic processing on the signals to obtain 512 pieces of line data over the circumferential direction. As a result, the image processing apparatus 1 acquires an ultrasound tomographic image illustrated on the left side of FIG. 3.

The image processing apparatus 1 arranges each line data radially with the ultrasound transmitter and receiver at the center position, thereby generating an ultrasound cross-sectional image of an orthogonal coordinate system illustrated on the right side of FIG. 3. The line data is dense at the center position of the rotation, and becomes sparse with distance from the rotation center. The image processing apparatus 1 generates pixels between lines by interpolating a region between the lines in the line data by known interpolation processing. As a result, a cross-sectional image in a direction orthogonal to the axial direction of the blood vessel can be generated as illustrated on the right side of FIG. 3.

The above-described processing is also applied to the optical transmitter and receiver (OCT). The optical transmitter and receiver emits near infrared rays a plurality of times during one rotation, and receives reflected waves. The image processing apparatus 1 generates an optical coherence cross-sectional image of a blood vessel based on the acquired line data.

When generating the cross-sectional image as described above, the image processing apparatus 1 according to the present embodiment performs processing of adding or embedding meta-information related to an image to a part of the cross-sectional image. Specifically, the image processing apparatus 1 adds information regarding an acquisition timing of acquiring the cross-sectional image to a portion located inside the sheath 41.

In FIG. 3, a portion located inside the sheath 41 is indicated by a thick frame. This portion is a header portion of the line data and is a portion unnecessary for an image observation since it is inside the sheath 41. Therefore, in the present embodiment, the information regarding the acquisition timing of acquiring the image is embedded in this portion, by which the image processing apparatus 1 is synchronized with the fluoroscopic imaging apparatus 2 while saving a band required for transferring the image to another apparatus.

The information regarding the acquisition timing is, for example, real time clock (RTC) information (or a time stamp) indicating a current time at the time of acquiring line data. At the time of acquiring the line data, the image processing apparatus 1 adds RTC information indicating the current time measured by the timer unit 16 to the header portion of the line data.

Alternatively, the information regarding the acquisition timing may be encoder information indicating an encoder pulse signal transmitted from the MDU 3 to the catheter 4. As described above, when driving the ultrasound transmitter and receiver and the optical transmitter and receiver, the MDU 3 outputs the encoder pulse signal to control the timings of transmitting ultrasound waves and near infrared rays. The image processing apparatus 1 may add encoder information indicating the encoder pulse signal to the line data as information indicating the acquisition timing.

As described above, the image processing apparatus 1 adds the meta-information to the header portion of the line data located inside the sheath 41. In this case, the image processing apparatus 1 corrects the meta-information based on the time required for predetermined signal processing performed on the line data, and then adds the meta-information to the line data.

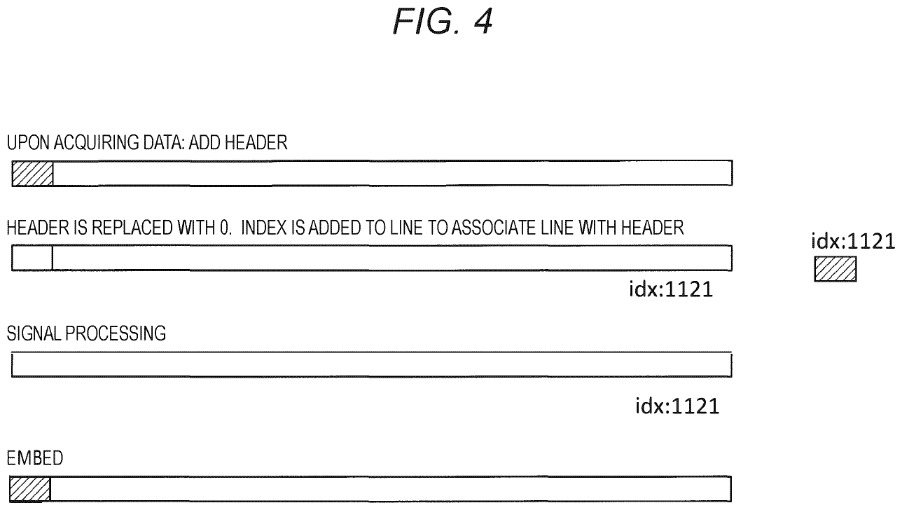

FIG. 4 is an explanatory diagram related to meta-information adding processing. FIG. 4 conceptually illustrates a change in line data before and after signal processing.

First, when acquiring line data from the catheter 4 via the MDU 3, the image processing apparatus 1 embeds meta-information in a header portion of the line data. Note that the image processing apparatus 1 may store the line data as raw data without any change.

Next, the image processing apparatus 1 separates the meta-information from the line data before performing signal processing on the line data. In this case, the header portion of the line data is replaced with 0. Index numbers for associating the line data and the separated meta-information with each other are respectively assigned to the line data and the separated meta-information.

Then, the image processing apparatus 1 performs predetermined signal processing on the line data. The signal processing is, for example, contrast adjustment, brightness adjustment, gamma correction, and the like of an image, but the details of the processing are not particularly limited.

Finally, the image processing apparatus 1 embed the temporarily separated meta-information within the header portion of the original line data based on the index numbers.

In this case, the image processing apparatus 1 corrects the meta-information (i.e., information regarding the acquisition timing) based on the time required for the signal processing, and then adds the meta-information to the line data. When signal processing is performed on the line data, the signal processing takes a certain time. In particular, it takes more time to generate an optical coherence cross-sectional image than to generate an ultrasound cross-sectional image. In view of this, in the present embodiment, meta-information corrected in consideration of the time required for the signal processing (i.e., the processing time is added to the original acquisition timing) is added, whereby images can be displayed in synchronization with each other in consideration of the time required for the signal processing when the images are finally displayed in synchronization with each other.

Returning to FIG. 3, the description will be continued. The image processing apparatus 1 generates a cross-sectional image of the blood vessel as illustrated on the right side of FIG. 3 by radially arranging a plurality of (e.g., 512) pieces of line data in the circumferential direction to which the meta-information is added. The image processing apparatus 1 displays the image in synchronization with another apparatus, i.e., the fluoroscopic imaging apparatus 2, on the basis of the meta-information regarding the acquisition timing. For example, the image processing apparatus 1 transfers the cross-sectional image to which the meta-information is added to the fluoroscopic imaging apparatus 2, and causes the fluoroscopic imaging apparatus 2 to synchronously display the transferred image based on the meta-information.

Note that, although the present embodiment has described the case where an image is displayed on the fluoroscopic imaging apparatus 2, the present embodiment is not limited thereto, and the image processing apparatus 1 may acquire a fluoroscopic image from the fluoroscopic imaging apparatus 2 and display the fluoroscopic image in synchronization with a cross-sectional image generated by the image processing apparatus.

As described above, according to the present embodiment, the meta-information regarding the acquisition timing is added to a part of the cross-sectional image, whereby synchronous display of images can be optimally achieved with the band required for transferring the cross-sectional image to another apparatus being saved.

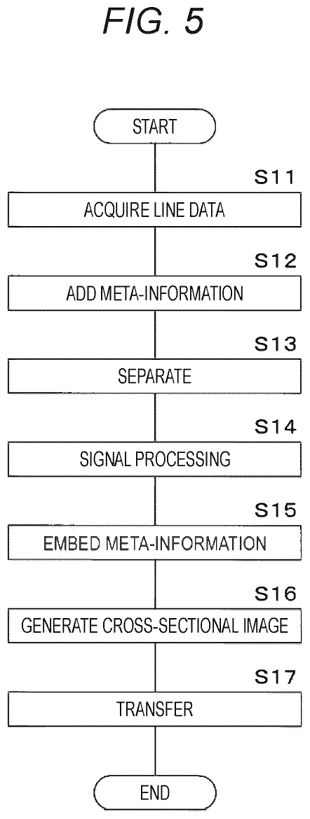

FIG. 5 is a flowchart of a processing procedure executed by the image processing apparatus 1. The details of the processing executed by the image processing apparatus 1 will be described with reference to FIG. 5.

The control unit 11 of the image processing apparatus 1 acquires, on the basis of reflected waves of ultrasound waves and reflected waves of near infrared rays emitted in the radial direction of the catheter 4 for a luminal organ like a blood vessel including the ultrasound transmitter and receiver and the optical transmitter and receiver on the distal end side, line data indicating the intensity with respect to the distance from the ultrasound transmitter and receiver and the optical transmitter and receiver (S11). The control unit 11 adds meta-information to a part of the acquired line data (S12). Specifically, the control unit 11 adds information regarding the acquisition timing of acquiring an image to a portion located inside the sheath 41 that covers the catheter 4. The information regarding the acquisition timing is, for example, RTC information indicating the current time measured by the timer unit 16, encoder information from the MDU 3, or the like.

The control unit 11 separates the meta-information from the line data before performing the signal processing (S13). The control unit 11 performs signal processing on the line data (S14). After performing the signal processing, the control unit 11 embeds the separated meta-information within the line data (S15). Specifically, the control unit 11 adds, after the signal processing, the information regarding the acquisition timing that has been corrected based on the time required for the signal processing.

The control unit 11 generates a cross-sectional image of the blood vessel from a plurality of pieces of line data over the circumferential direction (S16). The control unit 11 transfers the generated cross-sectional image to the fluoroscopic imaging apparatus 2 (S17), and ends the series of processing.

In the above description, the information related to the acquisition timing of acquiring the cross-sectional image is added as the meta-information, but the present embodiment is not limited thereto. For example, the image processing apparatus 1 may add information regarding a condition of capturing the cross-sectional image as the meta-information. As described above, the meta-information only needs to be metadata related to an image, and the detail thereof is not particularly limited.

Further, the case where the meta-information is added to a portion located inside the sheath 41 (i.e., the header portion of the line data) has been described above, but the present embodiment is not limited thereto. For example, the image processing apparatus 1 may add the meta-information to the outside of the line data. Alternatively, the image processing apparatus 1 may add the meta-information to a position of the line data farthest from the center of the image (e.g., a marginal portion that is not often used for display). Alternatively, the image processing apparatus 1 may collectively add the pieces of meta-information corresponding to all the pieces (512) of line data every time the image frame is switched. As described above, the image processing apparatus 1 only needs to be able to add the meta-information to a part of the cross-sectional image, and the place where the meta-information is added is not limited to the place located inside the sheath 41.

As described above, according to the present embodiment, information necessary for image synchronization with another apparatus can be embedded in an image.

It should be understood that the embodiments disclosed herein are illustrative in all respects and are not restrictive. The scope of the present invention should be defined by the claims rather than the above meaning, and is intended to include all conceivable modifications and variations within the meaning and scope equivalent to the claims.

Some or all of the subject matters described in the respective embodiments can be combined together. In addition, some or all of the independent claims and their dependent claims described in the claims can be combined together, regardless of their dependent relationships. Furthermore, the claims are described in a format (multi-claim format) in which a claim selectively recites two or more other claims. However, the claim format is not limited thereto. The claims may be described in a format (multi-multi claim) in which a multiple dependent claim is dependent on at least one multiple dependent claim.

What is claimed is:

1. A medical image processing system comprising:
a catheter rotatable and insertable into a blood vessel and including at least one of:
an ultrasound transceiver configured to transmit ultrasound waves and receive the waves reflected by the blood vessel in a radial direction of the catheter when the catheter is inserted in the blood vessel, and
an optical transceiver configured to emit near infrared rays and receive the rays reflected by the blood vessel in the radial direction when the catheter is inserted in the blood vessel;
a memory; and
a processor configured to execute a program that is stored in the memory to:
generate, based on the ultrasound waves received by the ultrasound transceiver or the near infrared rays received by the optical transceiver, a series of images each showing a location of a boundary of the blood vessel at a particular rotation angle of the ultrasound transceiver or the optical transceiver,
generate meta-information about the series of images,
embed the generated meta-information in the series of images, and
generate a cross-sectional image of the blood vessel showing the boundary using the series of images with the meta-information, and output the generated cross-sectional image.

2. The medical image processing system according to claim 1, wherein the meta-information embedded in the series of images indicates when the series of images is generated.

3. The medical image processing system according to claim 2, wherein the processor executes the program to acquire a timestamp from a real time clock (RTC) when the series of images is generated.

4. The medical image processing system according to claim 2, wherein the processor executes the program to update the meta-information when the cross-sectional image is generated.

5. The medical image processing system according to claim 4, wherein the updated meta-information indicates when the cross-sectional image is generated.

6. The medical image processing system according to claim 1, further comprising:
a drive device connectable to the catheter to rotate the catheter, wherein
the processor executes the program to acquire encoder information from the drive device and include the encoder information in the meta-information to be embedded in the series of images.

7. The medical image processing system according to claim 1, wherein the processor executes the program to perform image processing on the series of images and modify the meta-information based on a result of the image processing.

8. The medical image processing system according to claim 1, further comprising:
a display apparatus, wherein
the processor executes the program to cause the display apparatus to display the cross-sectional image according to the meta-information.

9. The medical image processing system according to claim 1, wherein the meta-information indicates an imaging condition under which the series of images is generated.

10. The medical image processing system according to claim 1, wherein
the catheter is covered with a sheath, and
the meta-information is embedded within a particular data area of the series of images corresponding to the sheath.

11. The medical image processing system according to claim 1, wherein the processor executes the program to:
separate the meta-information from the series of images,
perform signal processing on the series of images from which the meta-information is separated, and
restore the separated meta-information to the series of images after the signal processing is complete.

12. A method for processing a medical image captured through a catheter rotatable and insertable into a blood vessel and including at least one of:
an ultrasound transceiver configured to transmit ultrasound waves and receive the waves reflected by the blood vessel in a radial direction of the catheter when the catheter is inserted in the blood vessel, and
an optical transceiver configured to emit near infrared rays and receive the rays reflected by the blood vessel in the radial direction when the catheter is inserted in the blood vessel, the method comprising:
generating, based on the ultrasound waves received by the ultrasound transceiver or the near infrared rays received by the optical transceiver, a series of images each showing a location of a boundary of the blood vessel at a particular rotation angle of the ultrasound transceiver or the optical transceiver;
generating meta-information about the series of images;
embedding the generated meta-information in the series of images; and
generating a cross-sectional image of the blood vessel showing the boundary using the series of images with the meta-information, and outputting the generated cross-sectional image.

13. The method according to claim 12, wherein the meta-information embedded in the series of images indicates when the series of images is generated.

14. The method according to claim 13, further comprising:
acquiring a timestamp from a real time clock (RTC) when the series of images is generated.

15. The method according to claim 13, further comprising:
updating the meta-information when the cross-sectional image is generated.

16. The method according to claim 15, wherein the updated meta-information indicates when the cross-sectional image is generated.

17. The method according to claim 12, further comprising:
acquiring encoder information from a drive device configured to rotate the catheter, and including the encoder information in the meta-information to be embedded in the series of images.

18. The method according to claim 12, further comprising:
performing image processing on the series of images and modifying the meta-information based on a result of the image processing.

19. The method according to claim 12, further comprising:
displaying, on a display device, the cross-sectional image according to the meta-information.

20. A medical image processing apparatus comprising:
an interface circuit connectable to a catheter rotatable and insertable into a blood vessel and including at least one of:
an ultrasound transceiver configured to transmit ultrasound waves and receive the waves reflected by the blood vessel in a radial direction of the catheter when the catheter is inserted in the blood vessel, and
an optical transceiver configured to emit near infrared rays and receive the rays reflected by the blood vessel in the radial direction when the catheter is inserted in the blood vessel;
a memory; and
a processor configured to execute a program that is stored in the memory to:
generate, based on the ultrasound waves received by the ultrasound transceiver or the near infrared rays received by the optical transceiver, a series of images each showing a location of a boundary of the blood vessel at a particular rotation angle of the ultrasound transceiver or the optical transceiver,
generate meta-information about the series of images,
embed the generated meta-information in the series of images, and
generate a cross-sectional image of the blood vessel showing the boundary using the series of images with the meta-information, and output the generated cross-sectional image.
